(19) United States
(12) Patent Application Publication
(10) Pub. No.: US 2022/0296641 A1
(43) Pub. Date: Sep. 22, 2022

(54) DEAD SEA MINERAL BASED IMPLEMENTATION IN HIGH PERFORMANCE NONWOVEN FABRICS

(71) Applicants: AHAVA - DEAD SEA LABORATORIES LTD., Lod (IL); AVGOL INDUSTRIES 1953 LTD., Petah Tikva (IL)

(72) Inventors: Ariel YEDVAB, Kibbutz Ginegar (IL); Ma'or ZE'EVI, Dead Sea D.N Arvot Hayarden (IL); Meital PORTUGAL-COHEN, Jerusalem (IL); Eliran ISH-SHALOM, Kibbutz Tel Yosef (IL); Dror COHEN, D.N the Dead Sea (IL)

(73) Assignees: AHAVA - DEAD SEA LABORATORIES LTD., Lod (IL); AVGOL INDUSTRIES 1953 LTD., Petah Tikva (IL)

(21) Appl. No.: 17/596,067

(22) PCT Filed: Jun. 4, 2020
(86) PCT No.: PCT/IL2020/050624

§ 371 Date: Dec. 2, 2021

Related U.S. Application Data
(60) Provisional application No. 62/857,049, filed on Jun. 4, 2019.

(51) Int. Cl. A61K 35/08; A61L 15/18; A61L 15/44; A61K 8/96; A61K 8/02; A61Q 19/00; A61K 9/70; A61P 17/00; D04H 3/002; D01F 9/08

(52) U.S. Cl. CPC A61K 35/08; A61L 15/18; A61L 15/44; A61K 8/965; A61K 8/0208; A61Q 19/00; A61K 9/70; A61P 17/00; D04H 3/002; D01F 9/08

(57) ABSTRACT

Provided are nonwoven fabric materials including Dead Sea minerals which exhibit useful aesthetic and physical performance during the time of utilization in a consumer product. Further provided are processes for the preparation of the material and to uses thereof.

### TECHNOLOGICAL FIELD

This invention relates to nonwoven fabrics enriched with Dead Sea minerals, processes for the preparation thereof, uses thereof and products comprising same.

### BACKGROUND ART

Dead Sea minerals, such as salts and other mineral deposits taken from the Dead Sea have established benefits for skin health. Dead Sea minerals have been shown to comprise approximately 20 percent silicon dioxide, 15 percent calcium oxide, 5 percent aluminum oxide, 5 percent magnesium oxide, 3 percent iron oxide, 2 percent sodium oxide, 1 percent potassium oxide, and other minerals in lesser percentages [1]-[2].

Early prior art first address the means and methods of forming a basic spunmelt as exemplified by spunbond and meltblown nonwoven technologies), such as exemplified in publications [3]-[6]. Various methods of fabricating laminate materials into nonwoven fabrics include disclosure in the prior art of layered meltspun components mechanically engaged by application of hydraulic energy to influence filament displacement beginning with publications [7]-[11], disclose lay-down of multiple meltspun nonwoven fabrics materials or coforms with hydraulic energy used as a means of engaging said meltspun layers. Publication [12] presents high pressure means to attain total impact energies of 0.7 MJ-N/Kg to form an integrated web. Similarly, publication [13] offers means of fracturing filaments at self fused zones wherein total impact energies of 1.4 MJ-N/Kg or greater are used. Alternate means of filament control are disclosed in publication [14] wherein foraminous surfaces are used to support and direct filament movement under the influence of hydraulic energy. Publications [15] and [16] offer an approach wherein to attain suitable filament movement and integration it is necessary to have either a low thermal point bond of less than 10% of the material surface area or an anisoptropic bond pattern allowing for sufficient free filament length and engagement thereof. Publication [17] describes high performance filamentous material.

### REFERENCES

[1] Ma′or, Zeev et al., Antimicrobial properties of Dead Sea black mineral mud,

International Journal of Dermatology, May 2006, 45, 504-511.
[2] U.S. Pat. No. 9,693,936 to Magdassi et al.
[3] U.S. Pat. No. 3,849,241 to Butin, et al.
[4] U.S. Pat. No.3,855,046 to Hansen.
[5] U.S. Pat. No.4,041,203 to Brock, et al,
[6] U.S. Pat. No.7,611,594 to Sommer et al.
[7] U.S. Pat. No. 3,485,706 to Evans.
[8] U.S. Pat. No. 4,879,170 to Radwanski, et al.
[9] U.S. Pat. No. 4,931,355to Radwanski, et al.
[10] U.S. Pat. No. 4,950,531 to Radwanski, et al.
[11] U.S. Pat. No. 4,939,016 to Radwanski, et al.
[12] U.S. Pat. No. 5,023,130 to Simpson, et al.
[13] Japanese Patent Application.
[14] U.S. Pat. No. 6,321,425 to Putnam, et al.
[15] U.S. Pat. No. 7,858,544 to Puri, et al.
[16] U.S. Pat. No. 8,093,163 to Turi, et al.
[17] WO 2014/055098.
[18] U.S. Pat. No. 6,537,644 to Kauschke, et al.
[19] U.S. Pat. No. 6,610,390 to Kauschke, et al.
[20] U.S. Pat. No. 5,709,747 to Golswasser.
[21] U.S. Pat. No. 5,885,656 to Golswasser.

The content of each of the aforementioned publications is incorporated herein by reference.

Acknowledgement of the above references herein is not to be inferred as meaning that these are in any way relevant to the patentability of the presently disclosed subject matter.

### SUMMARY OF THE INVENTION

The benefits of Dead Sea salts and minerals for skin health and beauty are well established and formulations of Dead Sea different salts and minerals are widespread in modem cosmetics.

There remains however an unmet need for a nonwoven fabric containing Dead Sea minerals that exhibits functional and aesthetic/physical features appealing to end-use customers.

The inventors of the present invention have utilized sources of Dead Sea salts and minerals to enrich a filamentous material (e.g., a nonwoven fabric) with same.

The skin effectiveness of the Dead Sea salts and minerals enriched filamentous material was demonstrated by topical application of said filamentous material on human skin organ culture.

Accordingly, the present invention is directed to a construct comprising filamentary components and more particularly to a filamentous material exhibiting useful physical performance while retaining suitable attributes to allow for mechanical processing of that material into useful consumer products comprising Dead Sea minerals. The filamentous material includes at least one integrating network of essentially continuous filaments formed from at least one polymeric material. To said integrating network of continuous filaments is added at least one performance modifying filamentous component, wherein the addition and integration of the performance modifying filamentous component results in a composite material exhibiting a useful function of tactile and ductile softness while retaining finite control of fluids. Such finite fluid control includes management of both liquids and gases in the same composite while providing favorable consumer perceived “fabric” or “flannel” like characteristics as well as retaining attributes such as strength and elongation to allow for subsequent converting processes. Representative means and methods for fabricating such filamentous materials from the aforementioned integrating network and performance modifying filamentous components are provided herein.

Thus, the present invention provides in one of its aspects a construct comprising filamentary components and Dead Sea minerals.

In another one of its aspects the present invention provides a filamentous material comprising filamentary components and Dead Sea minerals

In another one of its aspects the present invention provides a filamentous material comprising at least one integrating network of essentially continuous filaments, at least one performance modifying filamentous component and Dead Sea minerals.

In another one of its aspects the present invention provides means for the production of the filamentous material as herein disclosed and exemplified.

In a further one of its aspects the present invention provides a method for the production of a filamentous material comprised of at least one integrating network of essentially continuous filaments, at least one performance modifying filamentous component and Dead Sea minerals, the method comprises integration of the continuous filament integrating network and the performance modifying filamentous component by the application of hydraulic energy, and implementation of the Dead Sea minerals thereto.

In a further one of its aspects the present invention provides a product comprising the filamentous material of the invention.

In vet a further one of its aspects the present invention provides a filamentous material for use in the manufacture of a product e.g., an article.

In another one of its aspects the present invention provides a filamentous material or a product comprising same for use in protecting and/or improving and/or rejuvenating the state of at least a region of a skin of a subject, and/or preventing and/or treating imperfections of at least a region of a skin of a subject.

Yet, in another one of its aspects the present invention provides a method of protecting and/or improving and/or rejuvenating the state of at least a region of a skin, and/or preventing and/or treating imperfections of at least a region of a skin, the method comprising application of the filamentous material of the invention or a product comprising same onto at least a region of a skin of a subject in need thereof.

In a further one of its aspects the present invention provides a filamentous material or a product comprising same for use in the treatment and/or prevention of one or more skin disease or skin disorder.

In another one of its aspects the present invention provides a method for treating and/or preventing of one or more skin disease and/or skin disorder, the method comprising application of the filamentous material of the invention or a product comprising same onto at least a region of a skin of a subject in need thereof.

Yet, in a further one of its aspects the present invention provides a filamentous material or a product comprising same for use in one or more of attenuation of skin irritation, attenuation of skin inflammation and skin calming.

In yet a further one of its aspects the present invention provides a method of one or more of attenuation of skin irritation, attenuation of skin inflammation and skin calming, the method comprising application of the filamentous material of the invention or a product comprising same onto at least a region of a skin of a subject in need thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a representative method of producing a filamentous material according to some embodiments of the invention.

FIG. 2 is a representative filamentous material according to some embodiments of the invention utilizing a contiguous thermal bond pattern comprising thermal point bonds induced by a plurality of individual contact elements on a contact surface whereby the distance between any two individual contact elements is less than 0.5 mm.

FIG. 3 is a representative filamentous material according to some embodiments of the invention depicting more closely repeating surface areas having reduced bonding therein.

FIG. 4 is a representative filamentous material according to some embodiments of the invention depicting a contiguous bonding pattern defined as a pattern of thermal bonds wherein the pattern is comprised of a first and a second repeating unit surface area.

FIG. 5 is an enlarged portion of the filamentous material illustrated in FIG. 4.

FIG. 6 is another enlarged portion of the filamentous material illustrated in FIG. 4.

FIG. 7 is a representative intra-structure micro-zone filamentous material according to some embodiments of the invention depicting a first antimicrobial layer, a spatial layer, and an antimicrobial inactivity component layer.

FIG. 8 illustrates Thermal Gravimetric Analysis (TGA) of a non-woven fabric according to some embodiments of the invention.

FIG. 9 illustrates Thermal Gravimetric Analysis (TGA) of a non-woven fabric impregnated with water-based brine solution of Dead Sea minerals (Osmoter) according to some embodiments of the invention.

FIG. 10 illustrates Thermal Gravimetric Analysis (TGA) of a non-woven fabric impregnated with oil-based dispersion of Dead Sea minerals (Crystal Osmoter) according to some embodiments of the invention.

FIG. 11 illustrates the effect of the Dead Sea salts enriched non-woven fabrics on skin viability according to some embodiments of the invention.

FIG. 12 illustrates the effect of the Dead Sea salts enriched non-woven fabrics on IL-1α secretion according to some embodiments of the invention.

### DETAILED DESCRIPTION OF EMBODIMENTS

Various embodiments will be detailed herein in connection with various aspects of the invention. It is noted that one or more embodiments may be applicable to one or more aspects of the invention disclosed herein above and below. It is further noted that one or more embodiments which are detailed in connection with one aspect of the invention e.g., the filamentous material of the invention, may also be applicable to the other aspects of the invention as detailed herein e.g., methods, processes, articles/products and uses.

In a first one of its aspect the present invention provides a construct comprising filamentary components and Dead Sea minerals.

In some embodiments the construct is a filamentous material.

In some embodiments the filamentous material comprises at least one integrating network of essentially continuous filaments.

In some embodiments the filamentous material comprises at least one integrating network of essentially continuous filaments formed from at least one polymeric material.

In some embodiments the at least one integrating network is an integrating network of continuous filaments.

In some embodiments the filamentous material further comprises at least one performance modifying filamentous component.

In some embodiments the filamentous material comprises at least one integrating network of essentially continuous filaments and at least one performance modifying filamentous component.

In some embodiments the at least one performance modifying filamentous component is formed from at least one polymeric material.

In some embodiments the filamentous material is a composite material.

In some embodiments the filamentous material comprises at least one integrating network of essentially continuous filaments formed from at least one polymeric material and at least one performance modifying filamentous component.

In some embodiments the filamentous material comprises at least one integrating network of essentially continuous filaments formed from at least one polymeric material and at least one performance modifying filamentous component formed from at least one polymeric material.

In some embodiments both the at least one integrating network of essentially continuous filaments and the at least one performance modifying filamentous component, are formed from at least one polymeric material.

Non limiting examples of suitable polymeric materials include thermal melt and thermoset polymers.

In some embodiments the polymeric material is a thermal melt plastic.

Non limiting examples of thermal melt plastics include polyolefins, preferably polypropylene or polyethylene.

Other polymers suitable for use include polyesters, such as polyethylene terephthalate; polyamides; polyacrylates; polystyrenes; thermoplastic elastomers, block polymers, polymer alloys; and blends of these and one or more of other known fiber forming thermoplastic materials.

In some embodiments the filamentous material is comprised of one or more substrate such as fiber and/or filaments formed of a thermoplastic component fiber used alone or in combination with other fibrous materials including natural materials (e.g., cotton and cellulose), thermoplastics, and thermosets.

In some embodiments the substrate is a nonwoven substrate.

In some embodiments the nonwoven substrate may further include other functional performance attributes including but not limited to antimicrobial, softness, conformability, friction reduction modifiers, friction increase modifiers, and other one or more skin wellness agents.

In some embodiments the filamentous material comprises a contiguous bonding pattern wherein said contiguous bonding pattern exhibits a pattern of thermal point bonds wherein the pattern is comprised of a first and a second repeating unit surface area.

In some embodiments the first and second repeating surface areas are proximal to one another such that pattern of repeating surface areas extending in both the machine direction of production (length) and the cross direction of production (width).

In some embodiments the first repeating unit surface area includes a thermal bond area of 1.) of at least 30% of the total area making up the first repeating unit surface area or 2.) a single bonding point extending completely though the machine direction, cross direction or combined machine and cross direction of the first repeating unit surface area.

In some embodiments the second repeating unit surface area comprises a thermal bond area of less than 10% of the total area making up the second repeating unit surface area.

In some embodiments the filamentous material comprises a contiguous bonding pattern wherein a first repeating unit surface area is comprised of a thermal point bonds induced by a plurality of individual contact elements on a contact surface whereby the distance between any two individual contact elements is less than 0.5 mm.

In some embodiments the filamentous material has retention of form and function when subjected to external forces, such as those imparted by stretching, loading, straining, wetting, or abrasion, whether such forces are of a singular, periodic, cyclical, or variable nature.

In some embodiments the filamentous material has finite fluid control wherein such control includes management of both liquids and gases in the same composite.

In some embodiments the filamentous material provides favorable consumer perceived “fabric” or “flannel” like characteristics.

In some embodiments the filamentous material exhibits attributes such as strength and elongation to allow and facilitate subsequent converting processes.

In some embodiments the filamentous material has a liquid absorbency in the absence of chemical modification in one or more elements of the continuous filament integrating network.

In some embodiments the filamentous material has a liquid absorbency in the absence of chemical modification in one or more elements of the performance modifying filamentous components.

In some embodiments the filamentous material has a liquid absorbency in the presence of chemical modification in one or more elements of either the continuous filament integrating network or the performance modifying filamentous components.

In some embodiments, the integrating network e.g., the continuous filament 5 integrating network, is comprised of a spunbond nonwoven material.

In some embodiments, the performance modifying filamentous components is comprised of a meltblown nonwoven material.

In some embodiments, the ratio by weight of the continuous filament integrating network to the performance modifying filamentous component is greater than or equal to 4:1.

In some embodiments, the ratio by weight of the continuous filament integrating network to the performance modifying filamentous component is greater than or equal to 5:1.

In some embodiments the filamentous material exhibits an air permeability of 250 l/sqm/sec or greater per gram/square meter material construct total or final weight.

In some embodiments the filamentous material exhibits a fiber volume, as defined by the basis weight divided by bulk, in the range of 0.05 milligrams/cubic centimeter to 0.40 milligrams/cubic centimeter.

In another one of its aspects the present invention provides means for the production of a filamentous material comprising at least one integrating network of essentially continuous filaments and at least one performance modifying filamentous component as herein disclosed.

In a further one of its aspects the present invention provides a method for the production of a filamentous material comprised of at least one integrating network of essentially continuous filaments, at least one performance modifying filamentous component and Dead Sea minerals, the method comprises integration of the continuous filament integrating network and the performance modifying filamentous component by the application of hydraulic energy, and implementation of the Dead Sea minerals thereto.

In some embodiments the filamentous material is produces by utilizing one or more chemical modifications and/or one or more mechanical modifications, alone or in combination, to create novel substrates utilizing Dead Sea minerals. To this end the method for producing the filamentous material further comprises utilizing one or more chemical modifications and/or one or more mechanical modifications, alone or in combination.

In some embodiments chemical modifications e.g., for improved Dead Sea mineral retention and distribution in either anhydrous or hydrous environments, include utilization of one or more of ionic surface modifiers, durable binders, cleavable binders, pH buffering agents, encapsulants, softness agents, and other nutritives including oils and emollients.

In some embodiments mechanical modifications e.g., for improved Dead Sea mineral retention and distribution in either anhydrous or hydrous environments, include utilization of one or more of crimping, embossing, hydraulic displacement, heating, cooling, compaction, lofting and three dimensional profiling.

In some embodiments the method is conducted in either anhydrous or hydrous environment.

In some embodiments the Dead Sea minerals may be implemented in topsheet based on methodology of fabric coating.

In some embodiments Dead Sea minerals may be implemented in topsheet based on methodology of fabric coating using a surfactant e.g., Silastol 163.

In some embodiments Dead Sea minerals may be implemented in Leg-cuff based on top coating technology.

In some embodiments the Dead Sea minerals may be implemented utilizing kiss-rolls mechanism.

In some embodiments the Dead Sea minerals may be implemented utilizing spraying mechanism.

In some embodiments Dead Sea minerals may be implemented to the filamentous material e.g., nonwoven fabrics, as water-based brine solution (e.g., Osmoter),

In some embodiments Dead Sea minerals are obtained from water-based brine solution (e.g., Osmoter).

In some embodiments Dead Sea minerals may be implemented to the filamentous material e.g., nonwoven fabrics, as oil-based dispersion (e.g., Crystal Osmoter), wherein the oil-based dispersion may optionally be diluted, prior to implementation, in an aqueous or other polar solution.

In some embodiments Dead Sea minerals are obtained from an oil-based dispersion e.g., Crystal Osmoter).

In some embodiments the method may further comprise applying drying means to thereby provide a substantially dry filamentous material.

Is some embodiments the Dead Sea minerals are coating the filamentous material.

Is some embodiments the Dead Sea minerals are impregnated in the filamentous material.

In some embodiments the Dead Sea minerals in non-woven topsheet forms unique sensorial fabric.

In some embodiments the method for the production of the filamentous material of the invention may be one continuous process e.g., substantially as disclosed herein.

In some embodiments the method for the production of the filamentous material of the invention may comprises on or more different individual process stages e.g., substantially as disclosed herein.

The filamentous material in accordance with present invention may be produced by various means such as those described in publication [17], the content of which is incorporated herein by reference.

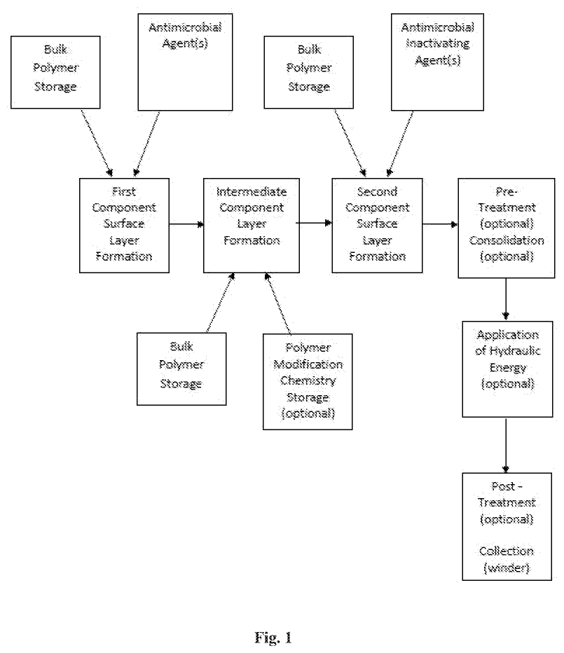

Representative methods of producing a filamentous material in accordance with present invention are depicted in FIG. 1 through FIG. 7. It should be noted that consolidation, pre-treatment by chemical or mechanical modification, and application of hydraulic energy may be effected by at various stages of lay-down of one or more integrating networks and one or more performance modifying filamentous components.

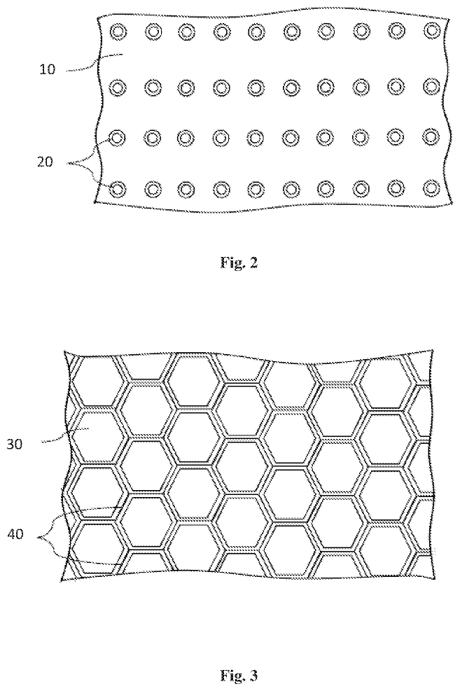

FIG. 2 is a representative filamentous material according to some embodiments of the invention utilizing a contiguous thermal bond pattern comprising thermal point bonds induced by a plurality of individual contact elements on a contact surface whereby the distance between any two individual contact elements is less than 0.5 mm.

FIG. 3 is a representative filamentous material according to some embodiments of the invention depicting more closely repeating surface areas having reduced bonding therein.

A representative means for production of an integrating network of continuous filaments includes those produced by spunbond nonwoven technology, though other woven, knitted or continuous spinning technologies are equally suitable. The spunbond continuous filaments used in the present invention have a basis weight of preferably at least about 3 gsm.

A process for the formation of spunbond involves supplying a molten thermal melt polymer, which is then extruded under pressure through a plate known as a spinneret or die head, The die head includes a spaced array of die orifices having diameters of generally about 0.1 to about 1.0 millimeters (mm). The resulting continuous filaments are quenched and drawn by any of a number of methods, such as slot draw systems, attenuator guns, or Godet rolls. The continuous filaments are collected as a loose web upon a moving collection surface, such as a wire mesh conveyor belt, When more than one spinneret is used in line for the purpose of forming a multi-layered fabric, the subsequent webs of filaments are collected upon the uppermost surface of the previously formed layer or web either continuously or in separately initiated batch processes.

The individual or combined layers or webs may be optionally consolidated at any step in the overall process, whether in an intermediate form or in a final, pre-conversion roll for, such as by means involving; 1.) heat and pressure, such as by thermal point bonding, 2.) application of hydraulic energy, such as by direct pressurized streams or is sprays of water, 3.) chemical bonding, such as by glues or adhesives, 4) thermal bonding, such as passage of elevated of elevated temperature air through the material, and 5.) combinations thereof. When a thermal point bond consolidation method is used, the web or layers of webs come into contact with a thermal conductive rolls, which may be either smooth or with an embossed pattern of individual contact elements to impart and achieve the desired degree of point bonding, usually on the order of 1 to 40 percent of the overall surface area being so bonded, These thermal point bonds may remain present in the final material, partially removed due to the application of a first degree of applied hydraulic energy, or essentially removed due to the application of a second degree of applied hydraulic energy. Further, the pattern or profile of the embossed roll may include a cross directional bias to the elements which impart the partial or complete consolidation of the fibrous components so as to alter the response of the fibrous components to force vector imparted by an applied hydraulic energy.

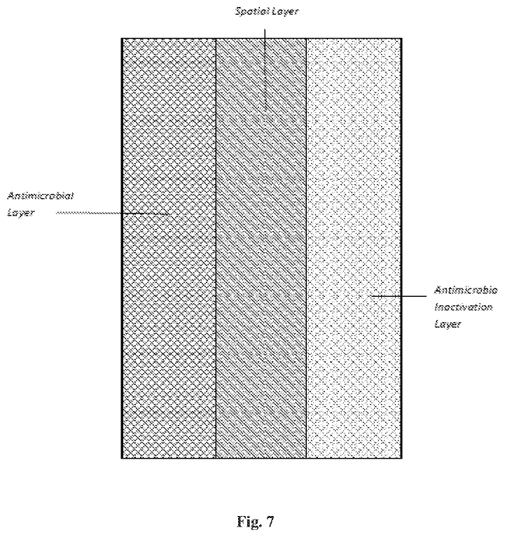

The formation of thermal point bonds by application of pressure and/or heat through direct contact of the integrating network of continuous filaments, the performance modifying filamentous component, or combinations thereof with one or more patterned rolls or rollers can exhibit particularly useful attributes in terms of both mode of integration and material formation, as well as resulting performance attributes in the finished article, Publications [18] and [19], hereby incorporated by reference in their respective entireties, in conjunction with the publications [15] and [16], direct their focus to fibrous materials exhibiting a defined nature of a bonding pattern to achieve a desired result (reference FIGS. 7, 8 and 9). Specifically, said publications disclose nonwovens having a non-symmetrical pattern of fusion bonds (that is, an anisotropic or asymmetrical pattern). As disclosed in these publications, bonds in an asymmetrical pattern may have a common orientation and common dimensions, yet define a total bond area along one direction (e.g., the MD) greater than along another direction (e.g., the CD) which is oriented orthogonally to the first direction, such that the points form a uniform pattern of bond density in one direction different from the uniform pattern of bond density in the other direction. Alternatively, as also disclosed in these publications, the bonds themselves may have varying orientations or varying dimensions, thereby to form a pattern of bond density which differs along the two directions. The bonds may be simple fusion bonds or closed figures elongated in one direction. The bonds may be closed figures elongated in one direction and selected from the group consisting of closed figures (a) oriented in parallel along the one direction axis, (b) oriented transverse to adjacent closed figures along the one direction axis, and (c) oriented sets with proximate closed figures so as to form therebetween a closed configuration elongated along the one direction axis.

While practice of an asymmetrical bonding pattern can he used to beneficially impact the production and performance of spunmelt nonwoven fabric, the inventors have found that similar or enhanced properties can be obtained through a contiguous bonding pattern methodology. A contiguous bonding pattern is defined as a pattern of thermal point bonds wherein the pattern is comprised of a first and a second repeating unit surface area. The first and second repeating surface areas are proximal to one another such that pattern of repeating surface areas extending in both the machine direction of production (length) and the cross direction of production (width). The first repeating unit surface area includes a thermal bond area of 1.) of at least 30% of the total area making up the first repeating unit surface area or 2.) a single bonding point extending completely though the machine direction, cross direction or combined machine and cross direction of the first repeating unit surface area. The second repeating unit surface area comprises a thermal bond area of less than 10% of the total area making up the second repeating unit surface area.

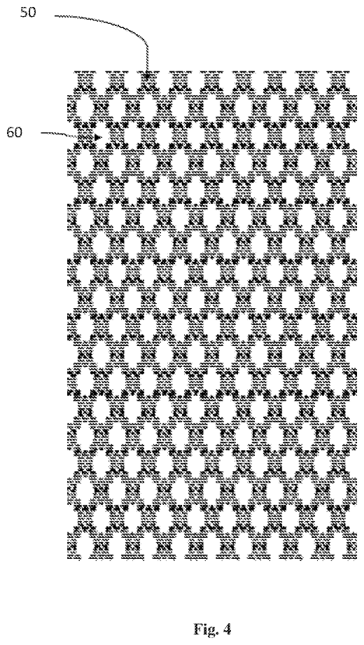

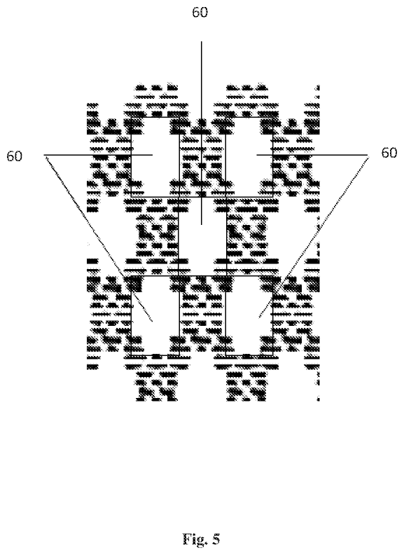

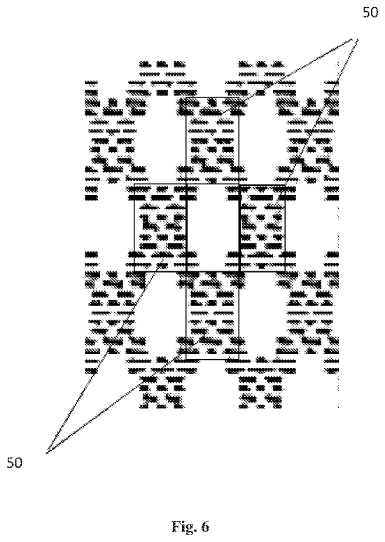

In the instance whereby the first repeating unit surface area is comprised of a thermal point bond of at least 30% of the total surface area of the first repeating unit surface area, the thermal point bond may be induced by a plurality of individual contact elements on a contact surface whereby the distance between any two individual contact elements is less than 0.5min, as represented by FIG. 4 through FIG. 6. As exemplified in FIG. 4, multiple thermal point bonds are induced in the first repeating unit surface area (“SA1”) 50 to create a higher degree of bonding than are created in the adjacent second repeating unit surface area (“SA2”) 60. It should be noted that the repeating unit surface area for SA1 and SA2 are defined as being rectilinear boundaries having the same total area. Further, it should be noted that a given SA1 will be circumscribed by a total of four (4) identical SA1 units, wherein each SA1 comes into contact with the vertex of an SA1 unit, (FIG. 6) and four (4) identical SA2 units, wherein each SA2 unit conies into contact with the side of an SA1 unit (FIG. 5). Conversely, it should be noted that a given SA2 will be circumscribed by a total of four (4) identical SA2 units, wherein each SA2 comes into contact with the vertex of an SA2 unit, and four (4) identical SA1 units, wherein each SA1 unit comes into contact with the side of an SA2 unit.

FIG. 7 is a representative intra-structure micro-zone filamentous material according to some embodiments of the invention depicting a first antimicrobial layer, a spatial layer, and an antimicrobial inactivity component layer. The filamentous material may further comprise optional secondary spatial layers (not depicted in the figure).

In some embodiments the filamentous material of the invention may have hybrid micro-zone which further includes a fluidic communication barrier layer.

In some embodiments the filamentous material of the invention may have hybrid micro-zone which further includes a fluidic communication barrier layer with partial and complete solubilization/dissolution mechanisms for infrastructure micro-zone creation.

In some embodiments the construct of the invention may comprise a separate antimicrobial functional article subsequently inserted into an antimicrobial inactivating envelope for disposal.

To further form the filamentous material of the present invention, the aforementioned integrating network of continuous filaments receives at least one performance modifying filamentous component, wherein the addition and integration of the performance modifying filamentous component results in a composite material exhibiting a useful function of tactile and ductile softness while retaining finite control of fluids. Such finite fluid control includes management of both liquids and gases in the same composite while providing favorable consumer perceived “fabric” or “flannel” like characteristics as well as retaining attributes such as strength and elongation to allow for subsequent converting processes.

Representative means and methods for fabricating such performance modifying filamentous components includes those produced by the meltblown nonwoven technology, though other technologies which produce fibrous elements of less than 10 micrometers in diameter, such as flash-spinning and nanofiber.

A representative meltblown process is similar in nature to the aforementioned lei spunbond process, which in place of essentially continuous filaments, this process involves the formation of discontinuous filamentary material. Again, a molten thermal melt polymer is extruded under pressure through orifices in a spinneret or die. High velocity air impinges upon and entrains the filaments as they exit the die. The energy of this step is such that the formed filaments are greatly reduced in diameter and are fractured so that microfibers of finite length are produced. This differs from the spunbond process whereby the continuity of the filaments is preserved. The process to form either a single layer or a multiple-layer fabric is continuous, that is, the process steps are uninterrupted from extrusion of the filaments to form the first layer until the bonded web is wound into a roll. Methods for producing these types of fabrics are described in publication [5] which is incorporated by reference in its entirety. The cross-sectional profile of individual elements within the integrating network or the performance modifying filamentous components is not a critical limitation to the practice of the present invention.

The individual elements within the integrating network or the performance modifying filamentous components may further be of homogenous or heterogeneous composition, include performance or aesthetic modifying melt additives, and be comprised of monocomponent, bicomponent, and/or multicomponent filament or fiber construction. Further, it is anticipated and within the purview of the present invention that one or more continuous filament integrating networks may he layered with one or more performance modifying filamentous components such that in the manufacturing or lay-down process: 1.) the components of each type alternate in order of lay-down; 2.) two or more layers of a component type are sequentially ordered for lay-down; 3.) an equal number of component type are used; 4.) and odd number of component types are used; 5.) the amount of component types are introduced in equal mass, composition or diameter; 6.) the amount of component types are introduced in different, varying, or incremental adjustment of introduced mass, composition, or diameter; and, 7.) combinations thereof. As mentioned previously, one or more consolidation step may be used between one or more lay-down steps in the manufacturing process.

Chemical based performance and/or aesthetic modifying melt additives includes those chemistries which result in modified properties of the filaments or fibers, such as to render the fibrous element hydrophobic, hydrophilic, enhance absorbency, render anti-static or flame retardant, modify crystallinity or strength, alter melt-flow rheology, and the like.

In some embodiments the filamentous material of the invention may be hydrophobic e.g., substantially not permeable to water and/or to water based solutions and/or to any other polar solutions, and/or substantially not having the ability to be wettened by water and/or by water based solutions and/or by any other polar solutions.

In some embodiments the filamentous material of the invention may be comprised of one or more hydrophobic fabrics.

In some embodiments the fabrics are non-woven fabrics.

In some embodiments the filamentous material of the invention may be hydrophilic e.g., substantially permeable to water and/or to water based solutions and/or to any other polar solutions, and/or substantially having the ability to be wettened by water and/or by water based solutions and/or by any other polar solutions.

In some embodiments the filamentous material of the invention may be comprised of one or more hydrophilic fabrics.

In some embodiments the fabrics are non-woven fabrics.

In some embodiments the filamentous material of the invention may be comprised of one or more hydrophobic fabric treated with an hydrophilic material e.g., hydrophilic finish material (e.g., Silastol 163) which imparts the filamentous material with hydrophilic characteristics.

The filamentous material in accordance with the present invention, including selective application to continuous filament integrating network elements, to performance modifying filamentous components, or precursor combinations thereof, are subjected to water jet treatment.

The water jet treatment allows for hydraulic energy to be imparted as a force on the elements in the filamentous material being produced. This hydraulic energy acts to displace or motivate elements with the filamentous material to inter-engage and form a composite performance, with such processes being known in the art as being “hydroentangled” or “hydroengorged”. Application of hydraulic enemy may occur upon either expansive plane or face of the filamentous material being produced and may occur in one or more sequential or alternating steps. The water jets are preferably present in an amount of 1-10 heads or manifolds per side and the water is provided at a pressure predetermined by the quality of the resultant fabric desired. Preferably the pressure of the water in the jets is in a range of about 50-about 400 bar per head, with the range of 100 to 300 bar being preferred. Unique to the produced filamentous material of the present invention, a high degree of integration is obtained wherein the fiber volume, as defined by the basis weight divided by bulk, in the range of 0.05 milligrams/cubic centimeter to 0.40 milligrams/cubic centimeter and exhibits an air permeability of 250 l/sqm/sec or greater per gram/square meter material construct total or final weight. Through a combination of manufacturing controls and specific management of the filamentary and fibrous composition, production and lay-down, the inventors have identified means by which to allow effective application of hydraulic energy to filamentous material having a low volume of filamentary and fibrous targets by which to impinge said hydraulic energy and induce movement by relative force vectors imparted thereby.

Following water jet treatment, and preferably before drying of the resultant filamentous material, the filamentous material can be treated with one or more chemical agents to further affect, e.g., enhance or modify, web secondary properties such as flame retardancy, anti-static nature, and the like. The chemical agents may be topically applied over the entire surface of the filamentous material or within preselected zones. These zones may be provided with the same surfactant or additive or a different surfactant or additive in order to provide zones with different or the same properties. An example of topical treatment suitable for use is described in publication [20] and [21].

A variation upon the topical treatment of the filamentous material is that the surfactants can be applied as an array or in discrete strips across the width of the filamentous material in order to create zone treatments to which different performance, functional and/or aesthetic properties can be provided.

The invention allows for the production of a filamentous material in one continuous process including various features to provide new or enhanced properties within the filamentous material, in particular with respect to absorbency and softness. However, the invention also allows for the production of the nonwoven filamentous material in different individual process stages, e.g., as a two or more step process wherein one is the manufacture of the integrating network of continuous filaments, one is the application or manufacture of performance modifying filamentous components and one involving hydraulic processing of the composite. This versatility allows for cost savings since a continuous line does not have to be provided in one place or utilized at one continuous time. For example, a composite including an integrating network and a performance modifying filamentous component can be produced and then wound for temporary storage before being subjected to water jet treatment. Further, the layers may be subjected to water jet treatment to provide for a filamentous material of the invention which is usable as such or may be placed in storage and subsequently treated based upon a desired end use for the filamentous material. This versatility provides for cost efficiency in terms of plant space required for the provision of equipment, versatility in the use of different equipment with respect to timing and products and the ability to provide filamentous material with varying properties based on the application to which the material will be put.

In a further one of its aspects the present invention provides a product comprising the filamentous material of the invention.

The filamentous material of the present invention exhibits retention of form and function when subjected to external forces, such as those imparted by stretching, loading, straining, wetting, or abrasion, whether such forces are of a singular, periodic, cyclical, or variable nature. This durability aspect of the filamentous material is useful in the making of numerous end-use consumer products, including but not limited to hygiene products, personal and surface wipes as well as medical products. Of particular importance, the durable aesthetic and physical performance relative to basis weight embodied by the inventive filamentous material offers desirable integration as one or more components of a diaper wherein use in affixation of the diaper to the wearer and/or skin contact and skin health properties when subjected to liquid insult suggestive of use in diaper constructs, are beneficial in view of the simultaneous presence of strength, elongation and low-linting performance that influence the materials convertibility by high-speed automated platforms and end use application.

In some embodiments the product is a personal skin care product a cleansing product and a moisturizing product).

In some embodiments the product is a medical product.

In some embodiments the product is a cosmetic product.

In some embodiments the product is a facial mask.

In some embodiments the product is a diaper.

Apparatus useful in preparing the filamentous material of the invention is conventional in nature and known to one skilled in the art. Such apparatus includes extruders, conveyor lines, water jets, rewinders or unwinders, topical applicators, calenders or compactors, and the like.

As used herein the term “Dead Sea minerals” refers to a mixture of natural minerals (including salts) obtained from the waters of the Dead Sea.

In some embodiments the waters of Dead Sea (at times referred to herein a “Dead Sea water” and abbreviated as DSW) refers to the saline waters obtained from the Dead Sea (Israel or Jordan) region or an aqueous solution prepared by dissolving Dead Sea minerals in an aqueous medium.

In some embodiments, the waters of Dead Sea is an aqueous solution which simulates such natural solution, namely an aqueous solution having salt and mineral content substantially identical to that measured for the natural DSW.

In some embodiments, the Dead Sea water may he obtained directly from the Dead Sea filtered water substantially having the same salt content (a hypersaline concentration) as that of the unfiltered Dead Sea water, or Dead Sea water treated by any one or more of various other methods employed to remove organic matter and residual contaminants therefrom.

In some embodiments, the Dead Sea water having:

1. a specific density of 1.25-1.35 g/ml,

2. pH=4.6-5.6 (at 25° C.), and/or

3. less than 100 cfu/g of non-pathogenic microbes.

The Dead Sea water having the above physical characteristics is a concentrated extract of Dead Sea water comprising (among other metal salt ions) Ca⁺², Mg⁺², Na⁺ and and high concentrations of anions such as Cl⁻ and Br⁻.

In some embodiments, the concentrations of these ions are, as assessed by a water analysis carried out by the Geological Survey of Israel:
Calcium (Ca²⁺): 35,000-40,000 mg/L
Chloride (Cl⁻): 320,000-370,000 mg/L
Magnesium (Mg⁺²): 92,000-95,000 mg/L
Sodium (Na⁺): 1800-3200 mg/L
Potassium (K⁺): 2,500 mg/L, and
Bromide (Br⁻): 10,000-12,000 mg/L.
   Other minerals may also exist in the waters.

In some embodiments, the Dead Sea Water comprises:
Calcium (Ca^{'2}): 35,000-40,000 mg/L
Chloride (Cl⁻): 320,000-370,000 mg/L
Magnesium (Mg⁺²): 92,000-95,000 mg/L
Sodium (Na⁺): 2400-3200 mg/L
Potassium (K⁺): 2,500 mg/L, and
Bromide (Br⁻): 10,000-12,000 mg/L.
   Other minerals may also exist in the waters.

In some embodiments, the Dead Sea Water comprises:
Calcium (Ca⁺²): 5,000-10,000 mg/L
Chloride (Cl⁻): 315,000-360,000 mg/L
Magnesium (Mg⁺²): 100,000-150,000 mg/L
Sodium (Na⁺): 1800-2200 mg/L
Potassium (K⁺): 1,000-2,000 mg/L, and
Bromide (Br⁻): 5,000-10,000 mg/L.
   Other minerals may also exist in the waters.

In some further embodiments, the Dead Sea Water comprises:

| | |
|---|---|
| Calcium (Ca⁺²) | 34,000-40,000 mg/L |
| Chloride (Cl⁻) | 320,000-370,000 mg/ L |
| Magnesium (Mg⁺²) | 90,000-95,000 mg/L |
| Potassium (K⁺) | 1,300-2,200 mg/L |
| Sodium (Na⁺) | 1,500-2,800 mg/L |
| Bromide (Br⁻) | 11,000-15,000 mg/L. |

Other minerals may also exist in the waters.

In some embodiments, the Dead Sea Water comprises:
Calcium (Ca⁺²): 38,000 mg/L
Chloride (Cl⁻): 345,000 mg/L
Magnesium (Mg⁺²): 92,500 mg/L
Sodium (Na⁺): 2000 mg/L
Strontium (Se⁺²): 800 mg/L
Potassium (K⁺): 1,400 mg/L, and
Bromide (Br⁻): 11,500 mg/L.
   Other minerals may also exist in the waters.

In some embodiments, the Dead Sea Water comprises:
Calcium (Ca⁺²): 38,000 mg/L
Chloride (Cl⁻): 345,000 mg/L
Magnesium (Mg⁺²): 92,500 mg/L
Sodium (Na⁺): 2000 mg/L
Strontium (Sr⁺²): 800 mg/I
Potassium (K⁺): 1,400 mg/L, and
Bromide (Br⁻): 11,500 mg/L.
   Other minerals may also exist in the waters.

In some embodiments, the DSW is a clear colorless viscous liquid (at 25° C.),

In some embodiments, the DSW is natural DSW which has undergone pre-treatment, e.g., having been concentrated by allowing water to evaporate, for example through solar evaporation, thereafter reconstituted to afford a solution.

In some embodiments the Dead Sea mineral are provided as water-based brine solution.

In some embodiments the water-based brine solution is Dead. Sea. Water preparation commercially available as “Mans Sal” or “Maris Aqua” (AHAVA, Israel) referred to herein below also as “Osmoter” or OSM.

In some embodiments the Dead Sea mineral are provided as oil-based dispersion. in some embodiments, the minerals are present in the dispersions in the form of nanoparticles.

In some embodiments the oil-based dispersion is Dead Sea mineral preparation commercially available as “Crystal-Osmoter” (AHAVA, Israel), referred to herein below also as C-OSM.

The Dead Sea mineral oil-based dispersion are generally produced in a process which involves formation of water-in-oil (W/O) emulsions; the water phase thereof comprises Dead Sea material, Dispersions of the Dead Sea materials in the oil phase are eventually obtained by subsequent evaporation of the water from the W/O emulsion.

The preparation of such Dead Sea mineral oil-based dispersions is described in publication [2], the content of which is incorporated herein by reference.

As noted above, the Dead Sea minerals enriched filamentous material of the present invention may be used in the preparation of various fabric containing products that may be in contact with a skin of a subject. Hence, the filamentous material of the present invention may serve as a vehicle to minerals providing beneficial effects on the skin upon contact therewith.

The minerals e.g., coating and/or impregnated in the filamentous material of the present invention may provide beneficial effect while brought into direct or indirect contact with the skin of a subject. A direct contact may be achieved while the minerals of the filamentous material are facing the skin of the subject and are in close contact therewith. An indirect contact may be when the fabric is structured in a product remote from a surface which is in direct contact with the skin. In such cases the contact may be mediated by other medium such as for example by other one or more fabrics e.g., when the filamentous material of the invention forms part of a diaper having further one or more fabric layers, one of same is in direct contact with the skin of a subject, and upon moistening of the fabric (e.g., upon urination in a diaper) diffusion of the salts/minerals via the one or more other fabric layers occurs and as such the salts reach the skin of the subject. To this end, said mediating medium is permeable to salts/minerals.

The invented Dead Sea based fabric when applied on skin presents health benefits as proven in biological skin models e.g. attenuation of irritation, inflammation and calming.

Accordingly, in a further one of its aspects the present invention provides a filamentous material or a product comprising same for use in one or more of attenuation of skin irritation, attenuation of skin inflammation and skin calming.

Yet, in a further one of its aspects the present invention provides a method of one or more of attenuation of skin irritation, attenuation of skin inflammation and skin calming, the method comprising application of the filamentous material of the invention or a product comprising same onto at least a region of a skin of a subject in need thereof.

Without wishing to be bound by theory, in some embodiments the hygroscopic characteristics of the Dead Sea minerals may provide the filamentous material of the present invention and the products comprising same the ability to be used to restore moisture e.g., to a skin of a subject once applied thereto.

In a further one of its aspect the present invention provides a filamentous material or a product comprising same for use in the treatment and/or prevention of one or more skin disease or skin disorder.

In another one of its aspects the present invention provides a method for treating and/or preventing of one or more skin disease and/or disorder, the method comprising application of the filamentous material of the invention or a product comprising same onto at least a region of a skin of a subject in need thereof.

In another one of its aspects the present invention provides a filamentous material or a product comprising same for use in protecting and/or improving and/or rejuvenating the state of at least a region of a skin of a subject, and/or preventing and/or treating imperfections of at least a region of a skin of a subject.

Yet, in another one of its aspects the present invention provides a method of protecting and/or improving and/or rejuvenating the state of at least a region of a skin, and/or preventing and/or treating imperfections of at least a region of a skin, the method comprising application of the filamentous material of the invention or a product comprising same onto at least a region of a skin of a subject in need thereof.

The term “skin” as used herein above and below refers to any part of the human or animal skin, including the whole surface thereof, hair and nails.

The terms “treatment” or “prevention” or any lingual variations thereof as used herein above and below refer to application of an effective amount of the Dead Sea minerals effective to ameliorate undesired symptoms associated with a skin disease/disorder, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The “effective amount”, whether therapeutically or cosmetically effective amount for purposes disclosed herein is determined by such considerations as may be known in the art. The amount of the Dead Sea minerals must be effective to achieve one or more of the above desired therapeutic or cosmetic effects, depending, inter aka, on the type and severity of the disease to be treated and the treatment regime. The effective amount is typically determined in appropriately designed clinical trials (dose range studies) and the person versed in the art will know how to properly conduct such trials in order to determine the effective amount.

In some embodiments the Dead Sea minerals are implemented on the filamentous as water-based brine solution, wherein said solution comprises at least about 0.1% Dead Sea water (e.g., Osmoter). At time said solution comprises about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4% and 2.5% (w/w) Dead Sea water (e.g., Osmoter). Any value which is between any one of the above values is within the scope of the present invention.

In some embodiments the Dead Sea minerals are implemented on the filamentous as oil-based brine dispersion (e.g., Crystal Osmoter), wherein said dispersion is provided in a water solution and the concentration of said dispersion in said water solution is at least about 0.1% (w/ w). At time the concentration of said dispersion in said water solution is about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4% and 2.5%. Any value which is between any one of the above values is within the scope of the present invention.

As used herein, AOL stands for All On Level, is the amount of the substantially dry Dead Sea minerals on the fabric and it is provided in w/w.

As used herein, “GSM” or “gr/m²” or “gsm” is the weight of the fabric in grains per square meter.

The weight of the filamentous material of the invention particularly when implemented with Dead Sea minerals, may play a role in the function and/or the processing thereof and the like.

In some embodiments the AOL is between about 0.1% to about 5% w/w, at times between about 0.1% to about 2.5% w/w, event at times between about 0.3% to about 2.5% w/w. In some embodiments the AOL is about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 2.8%, 4.9% and 5.0% (w/w). Any value which is between any one of the above values is within the scope of the present invention.

In some embodiments the AOL is about 0.3%.

In some embodiments the AOL is about 0.5%.

In some embodiments the gsm is at least about 3. In some embodiments the gsm is between about 7 to 200, at times between about 7 to about 100, event at times between about 7 to about 20. Any value which is between any one of the above values is within the scope of the present invention.

In some embodiments the gsm is about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0. Any value which is between any one of the above values is within the scope of the present invention.

In some embodiments the gsm is about 13.0. In some embodiments the gsm is about 13.5. In some embodiments the gsm is about 14.0.

As used herein above and below the term “about” refers to +10% of the indicated value.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

The terms “cosmetic product” or “skin care product” relate to a product that can be used topically by application to a skin region (without substantially inducing systemic effect, the skin region being any part of the human or animal skin, including hair and nails) for achieving a cosmetic benefit, hygiene or skin-care.

In some embodiments, the cosmetic products are for modulate or alleviate wrinkling, photo-damage, and dryness in the skin of a subject. The cosmetic products may additionally regulate skin condition and signs of skin aging (all perceptible manifestations as well as any other macro or micro effects) by regulating visible and/or tactile discontinuities in skin texture, including fine lines, wrinkles, enlarged pores, roughness and other skin texture discontinuities associated with aged skin with reduced irritation and dryness.

### DETAILED DESCRIPTION OF EMBODIMENTS

The following examples arc not in any way intended to limit the scope of the invention as claimed.

### Example 1: Dead Sea Water-Based Brine Solution

In the present disclosure a commercial preparation of a Dead Sea water referred to herein as “Osmoter” or “Osmoter™” or “Mineral Skin Osmoter” or “OSM” was used. The preparations is also known as “Maris Sal” or “Maris Aqua” (Dead Sea Water, DSW) (Source: Geological Survey—Ministry of National Infrastructures, State of Israel, especially for AHAVA-Dead Sea Laboratories CAS #INCI Monograph ID:11089).

The “Osmoter” solution has the following composition:

| | |
|---|---|
| Salt normality (N) | Salt normality (N) |

| | | | |
|---|---|---|---|
| Na | 0.118 (2.720 g/l) | Rb | 3.5 × 10⁻⁶ (<3 × 10⁻⁴ g/l) |
| K | 0.054 (2.100 g/l) | Sb | <1.6 × 10⁻⁷ (<2 × 10⁻⁵ g/l) |
| Ca | 0.873 (35.000 g/l) | Sr | 7.6 × 10⁻³ (0.670 g/l) |
| Mg | 3.815 (92.700 g/l) | V | <7.9 × 10⁻⁵ (<0.004 g/l) |
| Ba | 6.6 × 10⁻⁵ (0.009 g/l) | Th | <8.6 × 10⁻⁸ (<2 × 10⁻⁵ g/l) |
| Cd | <1.8 × 10⁻⁷ (<2 × 10⁻⁵ g/l) | U | <8.4 × 10⁻⁸ (<2 × 10⁻⁵ g/l) |
| Co | <3.4 × 10⁻⁵ (<0.002 g/l) | Zn | <3.06 × 10⁻⁵ (<0.002 g/l) |
| Cu | <3.15 × 10⁻⁵ (<0.004 g/l) | Cl | 9.75 (346 g/l) |
| Cr | <3.85 × 10⁻⁴ (<0.02 g/l) | Br | 0.175 (14 g/l) |
| Fe | <3.58 × 10⁻⁵ (<0.002 g/l) | B | 0.011 (0.120 g/l) |
| Li | 5.76 × 10⁻³ (0.040 g/l) | As | 2.7 × 10⁻⁵ (0.002 g/l) |
| Mn | 1.82 × 10⁻⁴ (0.010 g/l) | I | 6.30 × 10⁻⁷ (8 × 10⁻⁸ g/l) |
| Mo | <1.04 × 10⁻⁶ (<10⁻⁴ g/l) | SiO2 | <3.33 × 10⁻⁴ (<0.02 g/l) |
| Ni | <3.4 × 10⁻⁵ (<0.002 g/l) | SiO4 | <2.2 × 10⁻³ (<0.2 g/l) |
| Pb | <9.6 × 10⁻⁸ (<2 × 10⁻⁵) | | |

Solutions comprising Dead Sea Water were prepared by dilutions of the “Osmoter” preparation. Various concentrations of the “Osmoter” preparation were used e.g., 0.3% and 0.5% (w/w).

It is noted that the percentages of the “Osmoter” in the disclosed solutions are provided herein above and below in weight per weight ratio (w/w) i.e., the weight in grams of the “Osmoter” per 100 gram total weight of the solution.

### Example 2: Oil-Based Dispersion of Dead Sea Minerals

In the present disclosure commercial preparations of a Dead Sea minerals oil based dispersion referred to herein as “Crystal Osmoter” or “C-OSM” were used.

Exemplary contents of the “Crystal Osmoter” are provided in Table 1 and Table 2 below.

TABLE 1
Crystal Osmoter Formulation I (used
in the studies detailed herein below)

| | Ing. Content % |
|---|---|
| INCI & COLIPA Name | (w/w) |
| Simmondsia Chinensis (Jojoba) Seed Oil | 58.20000 |
| Cyclohexasiloxane | 9.33605 |
| Cyclopentasiloxane | 7.17945 |
| Maris Aqua (Dead Sea Water/Osmoter) | 6.57940 |
| Ethyl Macadamiate | 4.99250 |
| Cetyl PEG/PPG-10/1 Dimethicone | 4.72330 |
| Propanediol (Corn derived) | 3.84580 |
| Isodecyl Isononanoate | 1.50000 |
| Glycerin | 1.46730 |
| Diisobutyl Adipate | 0.80000 |
| Parfum (Fragrance) | 0.40000 |
| PVP | 0.35930 |
| Pentaerythrityl Tetra-di-t-butyl Hydroxyhydrocinnamate | 0.20020 |
| Hippophae Rhamnoides (Oblipicha) Fruit Oil | 0.20000 |
| Borago Officinalis Seed Oil | 0.10000 |
| Olea Europaea (Olive) Fruit Oil | 0.09490 |
| Aqua (Water) | 0.00920 |
| Tocopherol (Vitamin E) | 0.00510 |
| Hypericum Perforatum Flower/Leaf/Stem Extract | 0.00500 |
| Malic Acid | 0.00250 |

TABLE 2
Crystal Osmoter Formulation II

| | Ing. Content % |
|---|---|
| INCI & COLIPA Name | (w/w) |
| Simmondsia Chinensis (Jojoba) Seed Oil | 58.20000 |
| Cyclohexasiloxane | 7.98640 |
| Aqua (Mineral Spring Water) | 6.43375 |
| Maris Aqua (Dead Sea Water/Osmoter) | 5.62590 |
| Cyclopentasiloxane | 5.32530 |
| Ethyl Macadamiate | 4.99250 |
| Cetyl PEG/PPG-10/1 Dimethicone | 4.03970 |
| Propanediol (Corn derived) | 2.83190 |
| Isodecyl Isononanoate | 1.50000 |
| Glycerin | 1.25685 |
| Diisobutyl Adipate | 0.80000 |
| Parfum (Fragrance) | 0.40000 |
| Pentaerythrityl Tetra-di-t-butyl Hydroxyhydrocinnamate | 0.20020 |
| Hippophae Rhamnoides (Oblipicha) Fruit Oil | 0.20000 |
| Borago Officinalis Seed Oil | 0.10000 |
| Olea Europaea (Olive) Fruit Oil | 0.09490 |
| Tocopherol (Vitamin E) | 0.00510 |
| Hypericum Perforatum Flower/Leaf/Stem Extract | 0.00500 |
| Malic Acid | 0.00250 |

Solutions comprising oil dispersions of Dead Sea minerals were prepared by dilutions of the “Crystal-Osmoter” preparation. Various concentrations of the “Crystal-Osmoter” preparation were used e.g., 0.3% and 0.5% (w/w).

It is noted that the percentages of the “Crystal-Osmoter” in the disclosed solutions are provided herein above and below in weight per weight ratio (w/w) i.e., the weight in grams of the “Crystal-Osmoter” per 100 gram total weight of the solution.

### Example 3: Filamentous Materials

Filamentous materials were formed in accordance with the present invention and tested per the protocols or standards detailed in Table 3.

TABLE 3
Tested methods and protocols of the filamentous
materials of the invention

| Test | Test method |
|---|---|
| Basis weight [gsm] | Avgol |
| MD tensile [N/5 cm] | WSP 110.4(05) B |
| CD tensile [N/5 cm] | WSP 110.4(05) B |
| MD Elongation [%] | WSP 110.4(05) B |
| CD Elongation [%] | WSP 110.4(05) B |
| MD HOM [gf] | WSP 90.3(05) |
| CD HOM [gf] | WSP 90.3(05) |
| Strike through [sec] | WSP 70.3(05) |
| Rewet [gr] | WSP 80.10(05) |
| Run-off [%] | WSP 80.9 |
| Air permeability [1/sqm/sec] | WSP 70.1 (05) |
| Fabric thickness [mm] | ASTM D645 |
| MD linting E-side [gr] | WSP 400.0 |
| MD linting S-side [gr] | WSP 400.0 |

In the Examples detailed below fabrics of 14 gsm, 13.5 or 13 gsm were used.

The fabrics were either fabrics in which all layers of the fabric were produced by Spunbond technology (e.g., SB fabric with two layers) or fabrics that were produced from three layers, with two outer Spunbond layers and one inner layer, that was produces by Meltblown technology (e.g., SMS fabrics).

The densities of the 3 layers for the 14 gsm SMS fabric were 5.60, 2.80, and 5.60 gsm, respectively.

The fabric were either hydrophilic or hydrophobic.

The hydrophilic fabrics were prepared from hydrophobic reference fabrics of the same density that were first treated with the hydrophilic finish Silastol 163 [Silastol 163 AOL was 0.7% (average of 3 points, automated determination of the spin finish level by Lenzing Instruments ALFA300 equipment] followed by treatment with the Dead Sea minerals.

Provided herein are exemplary 9 produced fabrics:

Sample 1: 14 gsm SMS, hydrophobic.
Sample 2: 14 gsm SMS, hydrophobic—was treated with Crystal-Osmoter (oil with dead sea minerals), Crystal-Osmoter AOL (Add On Level) was 0.3% (average of 3 points, Determination of isopropanol extractable finish level) (also referred to as C.OSM 0.3%). Strike Through, Rewet results—Not relevant (hydrophobic sample). This sample was prepared by treatment with a solution of Crystal-Osmoter in Ethanol (20% w/w), application. Treatment can also be done with 100% w/w Crystal Osmoter.
Sample 3: 14 gsm SMS. hydrophobic—was treated with Crystal-Osmoter. Crystal-Osmoter AOL was 0.5% (average of 3 points, Determination of Isopropanol extractable finish level) (also referred to as C.OSM 0.5%). Strike Through, Rewet results—Not relevant (hydrophobic sample). This sample was prepared by treatment with a solution of Crystal-Osmoter in Ethanol (20% w/w), kiss-roll application. Treatment can also be done with 100% w/w Crystal Osmoter.
Sample 4: 14 gsm SMS, hydrophilic (Silastol 163, 0.7%), Silastol 163 AOL was 0.7% (average of 3 points, Automated determination of the spin finish level by Lenzing Instruments ALFA300 equipment).
Sample 5: 14 gsm SMS, hydrophilic (Silastol 163, 0.7%)—was treated with Crystal Osmoter. Silastol 163 AOL was 0.7% (average of 3 points, Automated determination of the spin finish level by Lenzing Instruments ALFA300 equipment). Crystal-Osmoter AOL was 0.3% (average of 3 points, Determination of Isopropanol extractable finish level). This sample was prepared by treatment with a solution of Crystal-Osmoter in Ethanol (20% w/w), kiss-roll application. Treatment can also be done with 100% w/w Crystal Osmoter. Strike Through, Rewet results—Improvement in the results of single strike-through test and 1^{st} insult of the multiple strike-through test comparing to those of the hydrophilic reference sample (Sample 4). Deterioration in 2^{nd} and 3^{rd} insults of the multiple strike through results, for some of the test iterations (for the rest of the iterations results were about the same as those of the hydrophilic reference sample). Rewet results were about the same as those of the hydrophilic reference sample.
Sample 6: 14 gsm SMS, hydrophilic (Silastol 163, 0.7%)—was treated with Crystal Osmoter, Silastol 163 AOL was 0.7% (average of 3 points, Automated determination of the spin finish level by Lenzing Instruments ALFA300 equipment). Crystal-Osmoter AOL was 0.5% (average of 3 points, Determination of Isopropanol extractable finish level). This sample was prepared by treatment with a solution of Crystal-Osmoter in Ethanol (20% w/w), kiss-roll application. Treatment can also be done with 100% w/w Crystal Osmoter. Strike Through, Rewet results—Improvement in the results of single strike-through test and 1^{st} insult of the multiple strike-through test comparing to those of the hydrophilic reference sample (Sample 4). Deterioration in 2^{nd} and 3^{rd} insults of the multiple strike through results, for some of the test iterations (for the rest of the iterations results were about the same as those of the hydrophilic reference sample). Rewet results were about the same as those of the hydrophilic reference sample.
Sample 7: 13 gsm SB, hydrophobic.
Sample 8: 13 gsm SB, hydrophobic—was treated with Crystal-Osmoter. Crystal-Osmoter AOL was 2.2% (average of 3 points, Determination of Isopropanol extractable finish level). Strike Through, Rewet results—Not relevant (hydrophobic sample). This sample was prepared by treatment with 100% w/w Crystal Osmoter, kiss-roll application.
Sample 9: 13 gsm SB, hydrophobic—was treated with Crystal-Osmoter. Crystal-Osmoter AOL was 3.8% (average of 3 points, Determination of Isopropanol extractable finish level). Strike Through, Rewet results—Not relevant (hydrophobic sample). This sample was prepared by treatment with a solution of Crystal-Osmoter in Ethanol (70% w/w), kiss-roll application. Treatment can also be done with 100% w/w Crystal Osmoter.

Table 4 below provides details on the aforementioned 9 exemplary fabrics that were produced:

TABLE 4
exemplary fabrics

| | Sample | | | | | | | | |
|---|---|---|---|---|---|---|---|---|---|
| Test | 1 | 2 | 3 | 4 | 5 | 6 | 7 | 8 | 9 |
| Basis weight | 14 | 14 | 14 | 14 | 14 | 14 | 13 | 13 | 13 |
| [gsm], 10 points | | | | | | | | | |
| average | | | | | | | | | |
| MD HOM [mN], | 10.1 | 8.2 | 7.1 | 9.0 | 7.6 | 8.1 | | | |
| 3 points average | | | | | | | | | |
| CD HOM [mN], | 5.7 | 3.8 | 3.3 | 4.8 | 3.1 | 3.2 | | | |
| 3 points average | | | | | | | | | |
| Strike through | NA | NA | NA | 4.35 | 3.71 | 3.51 | | | |
| [sec], WSP | | | | | | | | | |
| 70.3(05), 5 | | | | | | | | | |
| points average | | | | | | | | | |
| Multiple Strike- | NA | NA | NA | 1^{st}- 2.83 | 1^{st}- 2.75 | 1^{st}- 2.59 | | | |
| Through, WSP | | | | 2^{nd}- 3.05 | 2^{nd}- 3.53 | 2^{nd}- 3.49 | | | |
| 70.7 (05), 5 | | | | 3^{rd}- 3.17 | 3^{rd}- 5.32 | 3^{rd}- 4.09 | | | |
| points average | | | | | | | | | |
| Rewet [gr]- 5 | NA | NA | NA | 0.11 | 0.10 | 0.11 | | | |
| points average | | | | | | | | | |
| Run-off [%]-5 | NA | NA | NA | 14.89 | 7.81 | 5.79 | | | |
| points average | | | | | | | | | |
| HydroHead | 170.0 | 37.4 | 42.5 | NA | NA | NA | | | |
| [mbar], WSP | | | | | | | | | |
| 80.6 (05) pressure | | | | | | | | | |
| rate of 30 | | | | | | | | | |
| mbar/min, 3 | | | | | | | | | |
| points average | | | | | | | | | |

### Example 4: Thermogravimetric Analysis Assay and Strike Through Measurements

Thermal gravimetric analysis (TGA, a method of thermal analysis in which the mass of a sample is measured over time as the temperature changes) of the filamentous materials of the invention was conducted.

This measurement provides information about physical phenomena such as absorption, adsorption and desorption.

For this purpose, the non-woven fabrics of the invention were post treated as follows:

Hydrophilic Top sheet fabric (spunbond 13.5 gsm treated with Silastol 163 anionic surfactant) was coated with different adding levels of Dead Sea solutions by using spray system (0-0.5 mg/cm2 of solid salt).

The samples were coated, dried, weighted and strike through was measured.

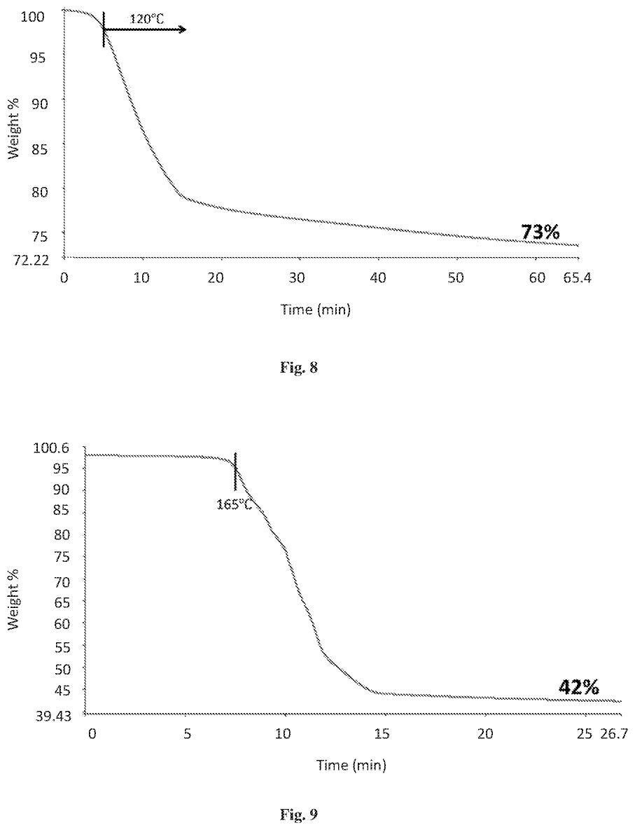

FIG. 8 illustrates TGA of a fabric that was not post treated with Dead Sea minerals. The fabric was heated to 120° C. at a heating rate of 25° C./min and remained at 120° C. for a time period of 1 hour at which the weight thereof was measured. After reaching 120° C. with TGA assay, the non-woven fabric weight reduced to 73% of its original weight the fabric lost 27% of its weight).

Strike throw i.e., the time (in sec) it takes to a 5 ml water to be absorbed in the fabric was 4.35 sec (tested with 14 gsm non-woven fabric).

FIG. 9 illustrates TGA of a fabric impregnated with the Osmoter water-based brine solution.

The fabric was heated to from 30° C. to 300° C. at a heating rate of 25° C./min and remained at 300° C. for 15 min. After reaching 300° C. with TGA assay, the weight of the non-woven fabric coated with the Osmoter water-based brine solution reduced to 42% of its original weight (i.e., the fabric lost 58% of its weight).

Without wishing to be bound by theory, the solids concentration in Dead Sea water, Osmoter, solution was about 30%. Given the limitations of the production process, complete drying of the Osmoter was nearly impossible even at heating to high temperature such as 300° C. compared to 120° C. in the fabric with no Dead Sea minerals. Even if the solution has been dried, it re-absorbed moisture from the air due to the hygroscopic nature thereof.

Strike through measurements of the fabric impregnated with the Osmoter water-based brine solution indicated that the strike through was not affected by coating fabrics with dead sea brine Osmoter (up to solids concentration of 0.5 mg/cm2).

Also, added weight for the fabrics with the Osmoter water-based brine solution was very high (due to moister part). Fabrics with high amount of solids felt wet and oily. These fabrics absorbed water from surrounding area and it turned heavy and wet (dripping water)

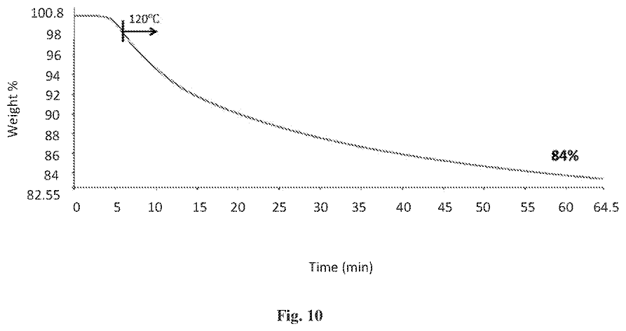

FIG. 10 illustrates TGA of a fabric post treated with the oil-based dispersion of Dead Sea minerals Crystal Osmoter. The fabric was heated to 120° C. at a heating rate of 25° C./min and remained at 120° C. for a time period of 1 hour at which the weight thereof was measured. After reaching 120° C. with TGA assay, the non-woven fabric weight reduced to 84% of its original weight, i.e., the fabric lost 16% of its weight compared to the Osmoter that lost much more.

Strike throw was 3.71 sec for 14 gsm non-woven fabric+0.86% AOL Crystal Osmoter. (the AOL value is an average of 3 points, determination of Isopropanol extractable finish level).

Strike throw was 3.51 sec for 14 gsm non-woven fabric+1.16% AOL Crystal Osmoter (the AOL value is an average of 3 points, determination of Isopropanol extractable finish level).

### Example 5: Skin Safety of Non-Woven Fabrics Implemented with Crystal Osmoter™ and Its Protection Against Irritation

The purpose of the study was to establish the safety and bio-activity of the non-woven fabrics of the invention implemented the Crystal Osmoter following topical treatment on human skin.

Bio-activity was evaluated on ex-vivo model using irritated Human Skin Organ Culture (HSOC). The irritation was achieved via skin exposure to the strong detergent sodium dodecyl sulfate (SDS). Skin irritation was measured using IL-1α bio-marker expression level and skin viability (MTT).

The following non-woven fabric samples were tested:
Crystal Osmoter in 14 gsm AOL—0.3% (average of 3 points, Determination of Isopropanol extractable finish level); and
Crystal Osmoter in 14 gsm AOL—0.5% (average of 3 points, Determination of Isopropanol extractable finish

The fabrics were treated with the Crystal Osmoter (100%) or with a solution of Crystal Osmoter in Ethanol (20% w/w) with kiss-roll application.

The following HSOC model was used: Human skin was obtained from 25-60-year-old healthy woman, skin type II who underwent abdominal plastic surgery, in accordance with the Declaration of Helsinki and the Hadassah University Hospital Ethics Committee approval (#0639-12-HMO). The skin was cleaned from underlying fat, cut into pieces of approximately 0.8×0.8 cm. It was then sterilized and cultured, with the dermal side down and the epidermal side exposed to air, in 6-well tissue culture plates containing DMEM, 100 U/ml penicillin, and 100 U/ml streptomycin at 37° C., under 5% CO₂.

Efficacy Evaluation:

On the HSOC ex-vivo epidermis a positive control for IL-1α secretion was topically applied with 3 μl of 5% the detergent SDS solution and the HSOC was incubate for 1 hour. After incubation on the HSOC a non-woven sheet, at the size of 0.8×0.8 cm², was laid down and incubated for 24 hours at 37° C. under 5% CO₂. After 24 hours of incubation, the medium of the HSOC ex-vivo was collected for the cytokine IL-1α ELISA and the HSOC ex-vivo was further processed for the viability assay.

Evaluation of IL-1α Cytokines Secretion:

IL-1α levels were assayed by ELISA kit (BioLegend, San Diego, Calif. USA). Briefly, ELISA maxisorb 96 well plates (NUNC, Roskilde, Denmark) were coated with a cytokine-specific capture antibody and incubated overnight at 4° C. The plates were washed three times (using PBS containing 0.05% Tween-20), blocking solution [PBS containing 1% (v/v) BSA] was added, and the plates were incubated for one hour at RI. Standards and samples were then introduced into the wells and incubated for 2 hours at room temperature (RI). The plates were then washed, and the detection of human IL-1α antibody was added for further incubation at RT for 1 hour. The plates were then washed and Avidin-horseradish peroxidase (HRP) was diluted 1:1000 and added. The plates were again incubated for 30 minutes at RT. The plates were then washed, and a substrate solution, TMB solution was added. Optical density, representing the number of viable cells, was read at 570 nm and 450 nm with a plate reader. The concentrations of IL-1α calculated based on the standard curve.

Evaluation of Cell Viability (MIT) Assay:

The epidermis was separated from the dermis by 1 minute of heating in phosphate-buffered saline (PBS) at 56° C. into heat-separated epidermal sheets. Using a 96 well plate, a 150 μl of 0.5 mg/mL Thiazolyl Blue Tetrazolium Bromide (MTT) diluted in PBS was added to each well. The plate was incubated at 37° C., 5% CO₂ for one hour. After incubation, the resulting precipitated stain was extracted in isopropanol with shaking for 15-30 minutes at room temperature. Optical density, representing the number of viable cells, was read at 570 nm with a colorimeter equipped with a Fluoroska™ Microplate (Thermo Fisher Scientific, Waltham, Mass., USA). The assay was performed in 4 replicates at least three times.

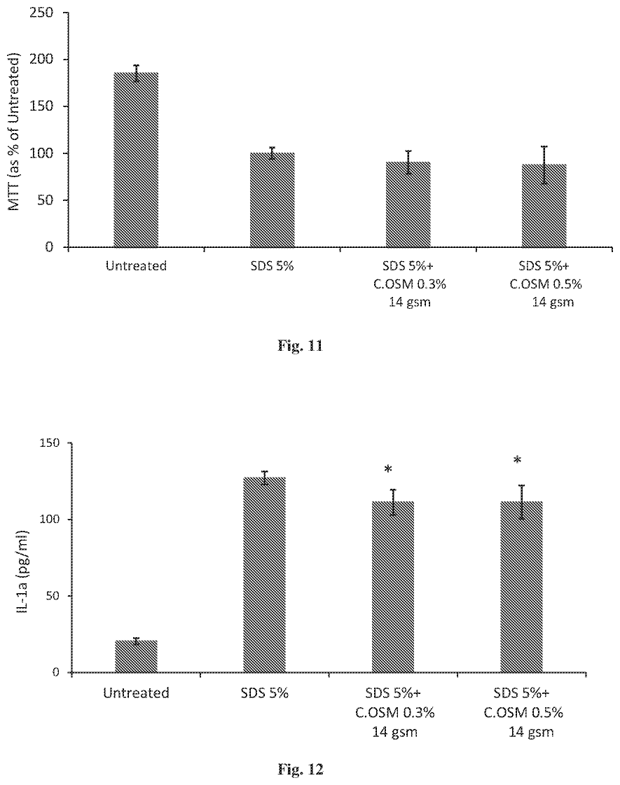

FIG. 11 illustrates the effect of the Crystal Osmoter enriched non-woven fabrics on skin viability. The viability assay (MTT) of HSOC was calculated by the % to untreated. The viability assay was measured after 24 of incubation from the SDS 5% allergenic agent application. p<0.05 vs. control. The values represent the mean±SEM. FIG. 11 represents the change in the viability between HSOC ex-vivo tissues that was exposed to 5% SDS treatment compared to 5% SDS+non-woven fabrics+Crystal Osmoter. As shown in FIG. 11, there was no negative effect on the cells viability of the fabrics topical treatment.

FIG. 12 illustrates the effect of the Crystal Osmoter enriched non-woven fabrics on IL-1α secretion, IL-1α was quantitated by ELISA assay, and the results are shown in (pg/ml) concentration. The IL-1α ELISA assay was measured after 24 incubation from 5% SDS application on the HSOC ex-vivo tissue. p<0.05 vs. control. The values represent the mean±SEM. FIG. 12 shows that a topical treatment of HSOC ex-vivo tissue to non-woven fabric+Crystal Osmoter resulted in a significant reduction of 10% in IL-1α level compared to skin treated only with 5% SDS.

The above results indicate that treatment with fabric implemented with Crystal Osmoter exhibited a significant protection against irritation, as indicated in FIG. 12 by a significant reduction of IL-1α—induced by irritation (SDS) by 10%. The viability of irritated HOSC epidermis was not negatively affected as illustrated in FIG. 11.

### Illustrative Embodiments

The following embodiments are illustrative and not intended to limit the claimed subject matter.
Embodiment 1 A filamentous material exhibiting useful physical performance while retaining suitable attributes to allow for mechanical processing of that material into useful products comprising Dead Sea minerals. The filamentous material includes at least one integrating network of essentially continuous filaments formed from at least one polymeric material. To said integrating network of continuous filaments is added at least one performance modifying filamentous component, wherein the addition and integration of the performance modifying filamentous component results in a composite material exhibiting a useful function of tactile and ductile softness while retaining finite control of fluids.
Embodiment 2 The filamentous material of Embodiment 1 produced by utilizing various chemical and mechanical modifications (alone or in combination) to thereby create novel substrates utilizing Dead Sea minerals.
Embodiment 3 The filamentous material of Embodiment 2, wherein the chemical modifications are for improved Dead Sea mineral retention and distribution in either anhydrous or hydrous environments and include ionic surface modifiers, durable binders, cleavable binders, pH buffering agents, encapsulants, softness agents, and other nutritives including oils and emollients.
Embodiment 4 The filamentous material of Embodiment 2 or 3, wherein the mechanical modifications include crimping, embossing, hydraulic displacement, heating, cooling, compaction, lofting and three dimensional profiling.
Embodiment 5 The filamentous material of any one of Embodiments 1 to 4, wherein the filamentous material is comprised of substrates including fiber and/or filaments formed of a thermoplastic component fiber used alone or in combination with other fibrous materials include natural materials (including but not limited to cotton and cellulose), thermoplastics, and thermosets.
Embodiment 6 The filamentous material of Embodiment 5, wherein said substrate is a nonwoven substrate which may further include other functional performance attributes including but not limited to antimicrobial, softness, conformability, friction reduction modifiers, friction increase modifiers, and other skin wellness agents.
Embodiment 7 The filamentous material of any one of Embodiments 1 to 6, wherein the Dead Sea minerals can be implemented in topsheet based on methodology of fabric coating using surfactant like Silastol 163.
Embodiment 8 The filamentous material of any one of Embodiments 1 to 6, wherein the Dead Sea minerals can be implemented in Leg-cuff based on top coating technology.
Embodiment 9 The filamentous material of any one of Embodiments 1 to 8, wherein the Dead Sea minerals can be implemented on nonwoven fabrics as water-based brine solution (Osmoter).
Embodiment 10 The filamentous material of any one of Embodiments 1 to 8, wherein the Dead Sea minerals can he implemented on nonwoven fabrics as oil-based dispersion (Crystal Osmoter).
Embodiment 11 The filamentous material of any one of Embodiments 1 to 10, wherein the Dead Sea minerals in non-woven topsheet forms unique sensorial fabric.
Embodiment 12 The filamentous material of any one of Embodiments 1 to 11, wherein said filamentous material forms a Dead Sea based fabric that when applied on skin presents health benefits as proven in biological skin models e.g. attenuation of irritation, inflammation and calming.
Embodiment 13 The filamentous material of any one of Embodiments 1 to 12, wherein the integrating network is a continuous filament integrating network and the integration thereof and the performance modifying filamentous component is by the application of hydraulic energy.
Embodiment 14 The filamentous material of any one of Embodiments 1 to 13, wherein the filamentous material comprises a contiguous bonding pattern wherein said contiguous bonding pattern exhibits a pattern of thermal point bonds wherein the pattern is comprised of a first and a second repeating unit surface area. The first and second repeating surface areas are proximal to one another such that pattern of repeating surface areas extending in both the machine direction of production (length) and the cross direction of production (width). The first repeating unit surface area includes a thermal bond area of 1.) of at least 30% of the total area making up the first repeating unit surface area or 2.) a single bonding point extending completely though the machine direction, cross direction or combined machine and cross direction of the first repeating unit surface area. The second repeating unit surface area comprises a thermal bond area of less than 10% of the total area making up the second repeating unit surface area.
Embodiment 15 The filamentous material of any one of Embodiments 1 to 14, wherein said filamentous material comprises a contiguous bonding pattern wherein a first repeating unit surface area is comprised of a thermal point bonds induced by a plurality of individual contact elements on a contact surface whereby the distance between any two individual contact elements is less than 0.5 min.
Embodiment 16 The filamentous material of any one of Embodiments 1 to 15, wherein said filamentous material having retention of form and function when subjected to external forces, such as those imparted by stretching, loading, straining, wetting, or abrasion, whether such forces are of a singular, periodic, cyclical, or variable nature.
Embodiment 17 The filamentous material of any one of Embodiments 1 to 16, wherein said having finite fluid control wherein such control includes management of both liquids and gases in the same composite.
Embodiment 18 The filamentous material of any one of Embodiments 1 to 17, wherein said filamentous material provides favorable consumer perceived “fabric” or “flannel” like characteristics.
Embodiment 19 The filamentous material of any one of Embodiments 1 to 18, wherein said filamentous material exhibits attributes such as strength and elongation to allow and facilitate subsequent converting processes.
Embodiment 20 The filamentous material of any one of Embodiments 1 to 19, wherein said filamentous material having a liquid absorbency in the absence of chemical modification in one or more elements of the continuous filament integrating network.
Embodiment 21 The filamentous material of any one of Embodiments 1 to 20, wherein said filamentous material having a liquid absorbency in the absence of chemical modification in one or more elements of the performance modifying filamentous components.
Embodiment 22 Means for the production of a filamentous material comprising an integrating network and performance modifying filamentous component e.g., the filamentous material of any one of Embodiments 1 to 21.
Embodiment 23 The filamentous material of any one of Embodiments 1 to 22, wherein said filamentous material having a liquid absorbency in the presence of chemical modification in one or more elements of either the continuous filament integrating network or the performance modifying filamentous components.
Embodiment 24 The filamentous material of any one of Embodiments 1 to 23, wherein the continuous filament integrating network is comprised of a spunbond nonwoven material.
Embodiment 25 The filamentous material of any one of Embodiments 1 to 24, wherein the performance modifying filamentous components is comprised of a meltblown nonwoven material.
Embodiment 26 The filamentous material of any one of Embodiments 1 to 25, wherein the ratio by weight of continuous filament integrating network to performance modifying filamentous component is greater than or equal to 4:1.
Embodiment 27 The filamentous material of any one of Embodiments 1 to 26, wherein the ratio by weight of continuous filament integrating network to performance modifying filamentous component is greater than or equal to 5:1.
Embodiment 28 The filamentous material of any one of Embodiments 1 to 27, wherein the filamentous material exhibits an air permeability of 250 l/sqm/sec or greater per gram/square meter material construct total or final weight.
Embodiment 29 The filamentous material of any one of Embodiments 1 to 28, wherein the filamentous material exhibits a fiber volume, as defined by the basis weight divided by bulk, in the range of 0.05 milligrams/cubic centimeter to 0.40 milligrams/cubic centimeter.

1-68. (canceled)

69. A filamentous material comprising filamentary components and Dead Sea minerals, wherein said filamentous material is a composite material.

70. The filamentous material of claim 69, comprising at least one integrating network of essentially continuous filaments and at least one performance modifying filamentous component.

71. The filamentous material of claim 70, wherein said at least one integrating network, at least one performance modifying filamentous component, or the combinations thereof, are formed from at least one natural material.

72. The filamentous material of claim 70, wherein said at least one integrating network, at least one performance modifying filamentous component, or the combinations thereof, are formed from at least one polymeric material.

73. The filamentous material of claim 72, wherein said polymeric material is selected from thermal melt and/or thermoset polymers.

74. The filamentous material of claim 73, wherein said thermal melt polymer is selected from polyolefins; polyesters; polyamides; polyacrylates; polystyrenes; thermoplastic elastomers, block polymers, polymer alloys; and blends thereof.

75. The filamentous material of claim 69, wherein said filamentary components further comprise other functional performance attributes, including antimicrobial, softness, conformability, friction reduction modifiers, friction increase modifiers, and the combinations thereof.

76. The filamentous material of claim 69, wherein said filamentous material comprises a contiguous bonding pattern wherein said contiguous bonding pattern exhibits a pattern of thermal point bonds.

77. The filamentous material of claim 76, wherein the thermal bond area is less than or equal to 10% of the total area.

78. The filamentous material of claim 69, wherein said filamentary components are hydrophobic or hydrophilic.

79. The filamentary components of claim 78, wherein said hydrophilic filamentary components exhibit an ability to absorb liquid, wherein said filamentous material exhibits a fiber volume, as defined by the basis weight divided by bulk, in the range of 0.05 milligrams/cubic centimeter to 0.40 milligrams/cubic centimeter.

80. The filamentous material of claim 69, wherein the Dead Sea minerals are incorporated into said filamentary components by topical coating, direct impregnation, and the combinations thereof.

81. A method for the production of a filamentous material comprised of at least one integrating network of essentially continuous filaments, at least one performance modifying filamentous component and Dead Sea minerals, wherein said method comprises combining said continuous filament integrating network and the performance modifying filamentous component by the application of energy, wherein said combined structure forms a contiguous structure of filamentous material.

82. The method of claim 81, wherein said energy is selected from thermal, hydraulic, mechanical and the combinations thereof.

83. The method of claim 80, wherein said Dead Sea minerals are applied by top coating, kiss-rolls, spraying, printing and the combinations thereof.

84. The method of claim 81, wherein the Dead Sea minerals are provided in a water-based brine solution or in an oil based dispersion form.

85. The method of claim 81, wherein the Dead Sea minerals are obtained by dissolution of said minerals into a solvent, optionally be further suspension thereof is a carrier.

86. A product comprising filamentous material produced by the method of claim 81, wherein the product is used as at least one component of personal care product.

87. A personal care product of claim 86 wherein the product is used for the attenuation of skin irritation, attenuation of skin inflammation, skin calming, treating skin ailments, preventing skin ailments, protecting skin, improving the healthy state of skin, preventing imperfections of the skin, treating imperfections, and the combinations thereof.

88. A personal care product of claim 87, where the product is applied to a defined region of skin.
